(19) United States
(12) Patent Application Publication
(10) Pub. No.: US 2008/0279796 A1
(43) Pub. Date: Nov. 13, 2008

(54) TRANSITION METAL-CONTAINING EFFECT PIGMENTS

(76) Inventors: Carsten HANDROSCH, Muhltal (DE); Frank Pflucker, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/042,517

(22) Filed: Mar. 5, 2008

(30) Foreign Application Priority Data
Mar. 5, 2007 (DE) 10 2007 010 986.7

(51) Int. Cl. A61K 8/19; C09B 67/08

(52) U.S. Cl. 424/63; 106/419; 106/439; 106/450; 106/453; 106/455; 106/456; 106/457; 106/418

(57) ABSTRACT

The present invention relates to effect pigments comprising transition-metal oxides or transition-metal hydroxyoxides or transition-metal hydroxides based on flake-form substrates which are uncoated or coated with one or more metal oxides, and to the use thereof, inter alia in paints, coatings, printing inks, plastics and in particular in cosmetic formulations.

The present invention relates to effect pigments comprising transition-metal oxides or transition-metal hydroxyoxides or transition-metal hydroxides based on uncoated or coated flake-form substrates, and to the use thereof, inter alia in paints, coatings, printing inks, plastics and in particular in cosmetic formulations.

Pigments containing a layer comprising an oxide/hydroxide of a transition-metal compound, such as, for example, Fe₂O₃, are employed as luster or effect pigments in many areas of industry, in particular in decorative coating, in plastics, paints, coatings, printing inks and in cosmetic formulations.

In order to improve the properties, application media generally comprise a number of additives, such as, for example, plasticizers, fillers, stabilizers, anti-ageing agents, lubricants and release agents, antistatics and colorants. An undesired interaction is frequently observed here, in particular, between the effect pigments on the one hand and additives in the application medium on the other hand, which is thought to involve transition-metal cations reacting with the organic-based additives. Thus, it is frequently observed in plastics that the stabilizer and/or ageing agent molecules diffuse to the surface of the pigment particles, where they result in a yellowing reaction, which frequently also proceeds in the dark, in particular if the plastics comprise phenolic components as anti-oxidants, heat stabilizers or UV stabilizers.

Plastics having phenolic constituents exhibit yellowing from a pigment concentration as low as 0.01% by weight. In particular if sterically readily accessible phenol compounds are present in the plastic, the yellowing reaction may already be evident during processing. In the case of sterically poorly accessible compounds, by contrast, the yellowing sometimes only occurs 18 months after processing. In general, the yellowing reaction is visible with the eye within 2 h in the case of processing at 80° C. and at a pigment concentration of, for example, 0.1% by weight. The yellowing reaction leads to unattractive effects, in particular, in the case of pigments having a relatively pale hue and considerably impairs the aesthetic impression of the plastic system. In the case of a very low concentration of the pigment, however, the yellowing also occurs in the case of darker hues. The cause of the yellowing reaction is frequently the photoactivity of the transition-metal layer consisting, for example, of TiO₂, Fe(OH)₃, FeOOH, Fe₂O₃, Fe₃O₄, CrO₃, ZnO or mixed oxides thereof, which often considerably accelerates the photolytic decomposition of the organic constituents in the plastic or coating.

WO 2006/018196 discloses specially post-coated interference pigments based on coated glass flakes, which have significantly lower photoactivity, for example compared with TiO₂-coated mica pigments. The pigments known from the prior art also significantly slow degradation of dihydroxyacetone (DHA) in cosmetic self-tanning formulations. However, the pigments known from the prior art are highly transparent interference pigments based on glass flakes with a TiO₂ coating, i.e. pigments with no inherent absorption color (“mass tone”). In cosmetics, however, it is also advantageous to have available effect pigments which combine an attractive beige to red-brown absorption color of iron-containing effect pigments with DHA stability. Cosmetic formulations comprising effect pigments containing an iron oxide or hydroxide layer generally have only very limited chemical stability owing to the chemical interaction of the pigment surface with the cosmetic active compounds, which, besides decomposition of the cosmetic active substance, also significantly minimizes the shelf life of the formulation.

The object of the present invention is therefore to find effect pigments having a transition-metal oxide or hydroxyoxide layer, in particular having an iron oxide- or iron hydroxyoxide-containing layer, which are substantially photochemically or thermochemically inactive on the surface and thus exhibit only a low tendency, or none at all, to form complexes with the organic substances in the formulations and, owing to their attractive beige to red-brown absorption color, can be employed as “skin-corrector” pigments in the cosmetic formulations.

Surprisingly, it has now been found that effect pigments based on flake-form substrates comprising an oxide/hydroxide of a transition metal can be stabilised if they have an SiO₂ and/or Al₂O₃ layer as outer layer. The encapsulation of the transition-metal oxide/hydroxide layer by means of SiO₂ and/or Al₂O₃ or their hydrates SiOₓ(OH)_{y}/AlOOH completely or substantially suppresses direct contact between chemically labile organic substances in the application media and the chemically reactive transition-metal oxide/hydroxide surface.

Owing to their mass tone, effect pigments of this type can, for example, be combined together with organic substances, such as, for example, dihydroxyacetone, cosmetic active compounds, such as, for example, UV filters, which are normally of only very limited chemical stability in the presence of transition-metal oxides, such as, for example, iron oxides or hydroxides, in cosmetic formulations, such as, for example, day creams, foundations or self-tanning creams. The shelf life with respect to destruction of the cosmetic active substance is significantly increased here by the use of the pigments according to the invention. Furthermore, very slow yellowing in coatings and plastics is observed in the case of pigmentation with the effect pigments according to the invention.

The present invention therefore relates to effect pigments based on uncoated or coated flake-form substrates, which are distinguished by the fact that they contain on the pigment surface, as the final layer, a layer package comprising
(A) a layer of iron oxide and/or iron oxohydrate and
(B) a layer of SiO₂ and/or Al₂O₃ or hydrates thereof,

or a layer package comprising
(A) a layer comprising a mixed oxide, hydroxide, oxohydrate or double hydroxide of the general formula

M¹ᵥM²_{w}Oₓ(OH)_{y}X_{z}, where
M¹=Fe and M²=Mg^{II}, Ca^{II}, Sr^{II}, Ba^{II}, Ti^{III/IV}, Zr^{IV}, Cr^{III}, Fe^{II/III}, Co^{II/III}, Ni^{II},
Cu^{II}, Ag^{I}, Zn^{II}, Al^{III}, Ga^{III}, Sn^{II/IV}, Sb^{III/IV}, Bi^{III},
X═Cl⁻, NO₃⁻, SO₄²⁻, CO₃²⁻ and
v=1-3; w=0-3; x=0-6, y=0-12 and z=0-1
where v+w>0 and x+y>0, and
v, w, x and y in the case of suboxides may also be non-integer coefficients, and
(B) a layer of SiO₂ and/or Al₂_{O}₃ or hydrates thereof.

The effect pigments according to the invention are particularly suitable for pigmenting plastics since the encapsulation of the transition-metal oxide/hydroxide layer with SiO₂ and/or Al₂O₃ means that they have significantly lower chemical reactivity at the surface and consequently, in particular with the phenolic constituents in the plastic, strongly suppress yellowing. Owing to their mass tone, the effect pigments according to the invention are very particularly suitable in cosmetic formulations which comprise organic substances, such as, for example, organic active compounds, UV filters, etc. The effect pigments according to the invention drastically increase the shelf life of formulations which comprise transition metal-sensitive organic substances.

The effect pigments according to the invention are furthermore very suitable for use as so-called “skin corrector” pigments, for example in ointments, creams and compact powders. “Skin corrector” pigments are generally distinguished by the fact that they are able to compensate for coloristic inhomogeneities of the skin (for example red spots, dark eye rings) by toning down the color inhomogeneities of the skin areas or ideally completely compensating for them through their inherent color. However, it is disadvantageous for this application if the characteristic luster of this class of pigments allows the skin to appear visually “greasy” under certain circumstances after application. This disadvantage is circumvented in the case of the effect pigments according to the invention in that the surface of the interference pigment is covered with iron ion-containing hydroxides or double hydroxides. These layers act as centres of scattering for the incident light and thus reduce the luster of the pigment without weakening the interference color so much that it would be disadvantageous for this application. In the case of iron ion-containing hydroxides or double hydroxides, it is in addition particularly advantageous that they contribute to the “skin corrector” pigment with an additional skin-, beige- to brown-colored absorptive color. The invention thus also relates to the use of the pigments according to the invention as “skin corrector” pigments.

Suitable base substrates for the effect pigments according to the invention are preferably transparent flakes, such as, for example, glass flakes, synthetic or natural mica flakes, SiOₓ flakes (x=≦2.0, preferably x=2), Al₂O₃ flakes, TiO₂ flakes, synthetic or natural iron oxide flakes, optionally passivated metal flakes, such as, for example, aluminium flakes, flakes of aluminium bronzes (i.e. copper alloys), brass bronzes, zinc bronzes, titanium bronzes or other comparable materials, graphite flakes, liquid crystal polymers (LCPs), holographic pigments, BiOCl flakes or mixtures of the said flakes. Preferred substrate mixtures are the following combinations:
synthetic or natural mica flakes+Al₂O₃ flakes
synthetic or natural mica flakes+glass flakes
synthetic or natural iron oxide flakes+synthetic or natural mica flakes
aluminium flakes or bronzes+synthetic or natural mica flakes
aluminium flakes or bronzes+Al₂O₃ flakes
aluminium flakes or bronzes+glass flakes
BiOCl flakes+glass flakes
BiOCl flakes+synthetic or natural mica flakes

The SiO₂ flakes preferably employed are synthetic, doped or undoped, preferably undoped, SiO₂ flakes which have a uniform layer thickness and are prepared, for example, on a continuous belt by solidification and hydrolysis of a water-glass solution, as described in WO 93/08237. Uniform layer thickness here is taken to mean a layer-thickness tolerance of 3 to 10%, preferably 3 to 5%, of the total dry layer thickness of the particles. The flake-form silicon dioxide particles are generally in amorphous form. Synthetic flakes of this type have the advantage over natural materials, such as, for example, mica, that the layer thickness can be set with respect to the desired effects and the layer-thickness tolerance is limited.

The very particularly preferred glass flakes can consist of all glass types known to the person skilled in the art, such as, for example, Ca/Al borosilicate glasses, window glass, C glass, E glass, ECR glass, Duran® glass, laboratory equipment glass, optical glass. Particular preference is given to E glass, ECR glass and Ca/Al borosilicate glasses. The refractive index of the glass flakes is preferably 1.45-1.80, in particular 1.50-1.70. Glass flakes of doped glass are furthermore suitable as preferred substrates. Suitable dopants are, for example, Fe, Bi, La, Nb, Ba, Ti, V, Ce, Au and Cu or mixtures thereof. Due to the doping, glasses having special properties, such as, for example, a high refractive index of up to 2.3 or strong inherent color, can be employed.

Suitable substrates are preferably flake-form substrates based on Ca/Al borosilicate (for example Ronastar® from Merck KGaA), SiO₂ (for example Color-stream® from Merck KGaA), Al₂O₃ (for example Xirallic® from Merck KGaA), natural leaf-form iron oxide (for example MIOX® from Kärntner Montan Industrie), graphite, synthetic flake-form iron oxide (for example TAROX® from Titan Kogyo) or metallic aluminium.

The substrate mixtures may also have the appearance that flakes having different particle sizes are mixed. In this case, they are preferably mixtures of mica flakes having different particle sizes, such as, for example, 1-15 μm, 1-25 μm, 3-8 μm, 3-10 μm, 5-25 μm, 5-30 μm, 5-50 μm, 17-26 μm, 10-60 μm, 5-100 μm, 10-100 μm, 10-125 μm, 10-150 μm, 20-180 μm, 20-200 μm, 45-500 μm. Through a suitable choice of the particle sizes in a mixture, the special properties and appearances of the product that are known to the person skilled in the art can be established specifically here. These are, for example,
high color brightness in mixtures of particles having a narrow particle size distribution;
combination of relatively high hiding power and sparkle in mixtures consisting of fine particles (preferably 1-25 μm) and coarse particles (preferably ≧100 μm, in particular 100-500 μm).

The size of the base substrates is not crucial per se and can be matched to the particular application. In general, the flake-form substrates have a thickness between 0.05 and 5 μm, in particular between 0.1 and 4.5 μm. Glass flakes preferably have a thickness of ≦1 μm, in particular ≦900 nm and very particularly preferably ≦500 nm. The size in the two other dimensions is usually between 1 and 250 μm, preferably between 2 and 200 μm, and in particular between 5 and 150 μm. The particle sizes are determined by means of laser diffraction on the powder or on pigment suspensions using instruments which are known to the person skilled in the art and are commercially available (for example from Malvern or Horiba).

The substrates preferably have a form factor (aspect ratio: diameter/thickness ratio) of 5-750, in particular of 10-300 and very particularly preferably of 20-200.

The base substrates may be uncoated or be mono- or multicoated with one or more, preferably colorless, metal oxides, in particular high-refractive-index metal oxides. Metal oxides which may be mentioned in particular are TiO₂ in the rutile or anatase modification, zirconium oxide, tin oxide, zinc oxide, silicon dioxide. The base substrates are preferably covered with one, two, three or four layers, in particular with one metal-oxide layer. If the base substrate has three oxide layers, they are preferably alternating high- and low-refractive-index layers, such as, for example, a TiO₂—SiO₂—TiO₂ layer sequence. Particularly preferred base substrates are natural and/or synthetic mica flakes, SiO₂ flakes, Al₂O₃ flakes, Fe₂O₃ flakes, glass flakes, passivated aluminium flakes, flakes made from aluminium bronzes, brass bronzes, zinc bronzes, titanium bronzes, or other comparable materials, TiO₂-coated mica, glass, SiO₂ or Al₂O₃ flakes, or TiO₂—SiO₂—TiO₂-coated mica, glass, SiO₂ or Al₂O₃ flakes, SiO₂—TiO₂-coated glass flakes, SiO₂—TiO₂—SiO₂—TiO₂-coated glass flakes, SiO₂-coated glass or Al₂O₃ flakes.
Particularly preferred substrates thus have the following structure:
substrate+high-refractive-index metal oxide+layer package (A)(B)
substrate+SiO₂+high-refractive-index metal oxide+layer package (A)(B)
substrate+layer package (A)(B)
substrate+SiO₂+layer package (A)(B)
substrate+high-refractive-index metal oxide+low-refractive-index metal oxide
   +high-refractive-index metal oxide+layer package (A)(B)
substrate+SiO₂+high-refractive-index metal oxide+low-refractive-index metal oxide+high-refractive-index metal oxide+layer package (A)(B)

The refractive index of the coated or uncoated substrates is preferably 1.20-2.20, in particular 1.50-1.70. The refractive index of the post-coated substrate is, however, not a parameter which is crucial per se. Thus, the stated ranges are also only intended to serve for explanation without representing a restriction. As in the case of metal flakes, substrates having a higher refractive index are naturally also suitable.

In this patent application, high-refractive-index coatings are taken to mean layers having a refractive index of >1.8, low-refractive-index layers are taken to mean those where n≦1.8.

If the substrate flake is covered with a TiO₂ layer, the TiO₂ can be in the rutile or anatase modification. It is preferably in the form of rutile. In this case, full-area or partial covering with SnO₂ or partial covering with SnO₂ nuclei is preferably carried out before the covering with TiO₂. This very thin SnO₂ layer has thicknesses of at most 10 nm, preferably ≦5 nm.

Layer (A) is preferably an iron oxide (hydroxide) layer, a mixed-oxide layer or a double hydroxide, in particular a layer of Fe₂O₃, Fe₃O₄, FeOOH, FeTiO₃, Fe₂TiO₅, of Fe(OH)₃/Ca(OH)₂ mixture, Fe(OH)₃/Al(OH)₃ mixture, Fe(OH)₃/Zn(OH)₂ mixture or Fe(OH)₃/Mg(OH)₂ mixture. The mixing ratio in the case of the hydroxide mixtures here can be between 0.1:99.9 and 99.9:0.1 parts by weight.

The thickness of layer (A) on the coated or uncoated substrate can of course be varied in broad ranges depending on the desired effect.

Layer (A) preferably has thicknesses of 2-350 nm, in particular of 5-200 nm. For the control of hue, luster and color strength, layer thicknesses of 20-150 nm are preferred.

The low-refractive-index coating (B) serves for encapsulation of layer (A) and preferably consists of SiO₂ and/or Al₂O₃. In the case of a mixture of SiO₂ and Al₂O₃, the mixing ratio is 1:100 to 100:1, preferably 1:50 to 50:1, in particular 1:10 to 10:1. Layer (B) can comprise 0.005-10% by weight, preferably 0.01-8% by weight, in particular 0.05-5% by weight, of further oxides or hydroxides from the group V, Zr, Zn, Ce, Ti, B, Na, K, Mg, Ca and/or Mn. Of the said oxides and hydroxides, particular preference is given to those of V, Zr, Ce and/or Zn. The thickness of the final layer (B) is, e.g., 2-200 nm, preferably 10-80 nm, in particular 10-60 nm.

In the case of particularly preferred effect pigments according to the invention, the proportion of layer (B) is 5-60% by weight, in particular 10-30% by weight and particularly preferably 12-20% by weight, based on the pigment as a whole.

The oil absorption value of the effect pigments according to the invention (determined in accordance with DIN EN ISO 787-5: 1995-10) is preferably 5-100, in particular 5-50 and very particularly preferably 20-40. However, the oil absorption value of the pigments according to the invention is not crucial per se.

The iron ion-containing pigments are distinguished not only by their attractive optical effects, but also exhibit a significantly improved shelf life in polymers, in particular in phenol-containing plastics and coatings. Furthermore, these pigments are distinguished by increased mechanical stability. Compared with effect pigments which do not have passivation by a layer (B), only slight paleadark yellowing or none at all is observed in the plastic, i.e. the pigments according to the invention exhibit only a slight surface reaction, if any, with the plastic components.

The invention furthermore relates to the use of the effect pigments according to the invention in paints, coatings, in particular automobile paints, industrial coatings, powder coatings, printing inks, security printing inks, plastics, ceramic materials, glasses, paper, in toners for electrophotographic printing processes, in seed, in greenhouse sheeting and tarpaulins, as absorbers in the laser marking of paper and plastics, as absorbers in the laser welding of plastics and in cosmetic formulations. The pigments according to the invention are furthermore also suitable for the preparation of pigment pastes with water, organic and/or aqueous solvents, pigment compositions and for the preparation of dry preparations, such as, for example, granules, chips, pellets, briquettes, etc. The dry preparations are particularly suitable for printing inks and in cosmetics.

Particularly preferred effect pigments according to the invention are listed below:

substrate+(SiO₂)+Fe₂O₃+SiO₂

substrate+(SiO₂)+TiO₂+Fe₂O₃+SiO₂

substrate+(SiO₂)+TiO₂+SiO₂+TiO₂+Fe₂O₃+SiO₂

substrate+(SiO₂)+FeOOH+SiO₂

substrate+(SiO₂)+TiO₂+FeOOH+SiO₂

substrate+(SiO₂)+Fe₂TiO₅+SiO₂

substrate+(SiO₂)+TiO₂+Fe₂TiO₅+SiO₂

substrate+(SiO₂)+FeTiO₃+SiO₂

substrate+(SiO₂)+TiO₂+FeTiO₃+SiO₂

substrate+(SiO₂)+Fe(OH)₃/Mg(OH)₂+SiO₂

substrate+(SiO₂)+TiO₂+Fe(OH)₃/Mg(OH)₂+SiO₂

substrate+(SiO₂)+Fe(OH)₃/Ca(OH)₂—+SiO₂

substrate+(SiO₂)+TiO₂+Fe(OH)₃/Ca(OH)₂+SiO₂

substrate+(SiO₂)+Fe₃O₄+SiO₂

substrate+(SiO₂)+TiO₂+Fe₃O₄+SiO₂

substrate+(SiO₂)+Fe(OH)₃/Al(OH)₃+SiO₂

substrate+(SiO₂)+TiO₂+Fe(OH)₃/Al(OH)₃+SiO₂

substrate+(SiO₂)+TiO₂+FeCa₂(OH)₆Cl+SiO₂

substrate+(SiO₂)+TiO₂+FeCa₂(OH)₆NO₃+SiO₂

substrate+(SiO₂)+TiO₂+FeMg₂(OH)₆CO₃+SiO₂

substrate+(SiO₂)+TiO₂+FeZn₂(OH)₆CO₃+SiO₂

substrate+(SiO₂)+TiO₂+CrO₃+Fe₃O₄+SiO₂

substrate+(SiO₂)+Fe(OH)₃/Zn(OH)₂+SiO₂

substrate+(SiO₂)+Fe(OH)₃/Zn(OH)₂+SiO₂/Al₂O₃

substrate+(SiO₂)+Fe₂O₃+SiO₂+Fe₂O₃+SiO₂/Al₂O₃

substrate+(SiO₂)+Fe₂O₃+SiO₂+Fe₂O₃+SiO₂

substrate+(SiO₂)+Fe₃O₄+SiO₂+Fe₃O₄+SiO₂/Al₂O₃

substrate+(SiO₂)+Fe₃O₄+SiO₂+Fe₃O₄+SiO₂

substrate+(SiO₂)+Fe₂O₃+SiO₂/Al₂O₃

substrate+(SiO₂)+TiO₂+Fe₂O₃+SiO₂/Al₂O₃

substrate+(SiO₂)+TiO₂+SiO₂+TiO₂+Fe₂O₃+SiO₂/Al₂O₃

substrate+(SiO₂)+FeOOH+SiO₂/Al₂O₃

substrate+(SiO₂)+TiO₂+FeOOH+SiO₂/Al₂O₃

substrate+(SiO₂)+Fe₂TiO₅+SiO₂/Al₂O₃

substrate+(SiO₂)+TiO₂+Fe₂TiO₅+SiO₂/Al₂O₃

substrate+(SiO₂)+FeTiO₃+SiO₂/Al₂O₃

substrate+(SiO₂)+TiO₂+FeTiO₃+SiO₂/Al₂O₃

substrate+(SiO₂)+Fe(OH)₃/Mg(OH)₂+SiO₂/Al₂O₃

substrate+(SiO₂)+TiO₂+Fe(OH)₃/Mg(OH)₂+SiO₂/Al₂O₃

substrate+(SiO₂)+Fe(OH)₃/Ca(OH)₂—+SiO₂/Al₂O₃

substrate+(SiO₂)+TiO₂+Fe(OH)₃/Ca(OH)₂+SiO₂/Al₂O₃

substrate+(SiO₂)+Fe₃O₄+SiO₂/Al₂O₃

substrate+(SiO₂)+TiO₂+Fe₃O₄+SiO₂/Al₂O₃

substrate+(SiO₂)+Fe(OH)₃/Al(OH)₃+SiO₂/Al₂O₃

substrate+(SiO₂)+TiO₂+Fe(OH)₃/Al(OH)₃+SiO₂/Al₂O₃

If the substrate is a glass or aluminium flake, the substrate is preferably covered directly with an SiO₂ layer. In the use according to the invention as “skin corrector” pigment, the substrate employed is preferably natural or synthetic mica and/or glass flakes, in particular TiO₂ (rutile)-coated natural or synthetic mica or glass flakes, optionally SiO₂-coated glass flakes.

The metal-oxide (hydroxide) layers are preferably applied by wet-chemical methods, it being possible to use the wet-chemical coating methods developed for the preparation of effect pigments. Methods of this type are described, for example, in DE 14 67 468, DE 19 59 988, DE 20 09 566, DE 22 14 545, DE 22 15 191, DE 22 44, 298, DE 23 13 331, DE 15 22 572, DE 31 37 808, DE 31 37 809, DE 31 51 343, DE 31 51 354, DE 31 51 355, DE 32 11 602, DE 32 35 017 or in further patent documents and other publications known to the person skilled in the art.

In the case of wet coating, the substrate particles are suspended in water, and one or more hydrolysable metal salts or a water-glass solution are added at a pH which is suitable for hydrolysis and which is selected in such a way that the metal oxides or metal oxide hydrates are precipitated directly on the flakes without secondary precipitations occurring. The pH is usually kept constant by simultaneous metered addition of a base and/or acid. The pigments are subsequently separated off, washed and dried at 50-150° C. for 6-18 h and, if the oxide layers are desired, calcined for 0.5-3 h, where the calcination temperature can be optimised with respect to the coating present in each case. In general, the calcination temperatures are between 500 and 1000° C., preferably between 600 and 900° C., depending on the substrate and coating. In the case of hydroxyoxides, such as, for example, FeOOH, and hydroxides/hydroxide mixtures, such as, for example, Fe(OH)₃/Mg(OH)₂ or FeCa₂(OH)₆NO₃, the calcination step is omitted and the pigments are only dried at max. 120° C. If desired or if necessary in the case of individual layer sequences (for example hydroxide layer on an oxide layer), the pigments can be separated off, dried and optionally calcined after application of individual coatings and then resuspended for precipitation of the further layers.

The precipitation of the SiO₂ layer on the substrate is generally carried out by addition of a potassium or sodium water-glass solution at a suitable pH.

The coating can furthermore also be carried out in a fluidized-bed reactor by gas-phase coating, it being possible to use correspondingly, for example, the methods proposed in EP 0 045 851 and EP 0 106 235 for the preparation of effect pigments.

The hue of the effect pigments according to the invention can be varied in very broad limits through the different choice of the layer compositions, layer combinations, the coating quantities or the layer thicknesses resulting therefrom. Fine tuning for a certain hue can be achieved here beyond the pure choice of amount by approaching the desired color under visual or measurement-technology control.

The absorptive color of the iron-containing coating of the interference pigment can be adjusted specifically by means of a plurality of adjustment levers. These are, for example:
precipitation of Fe(OH)₃ or FeOOH—the color of the pigment can be adjusted by means of the amount of precipitated hydroxide.
precipitation of Fe(OH)₃ or FeOOH in combination with white alkaline-earth metal, alkali metal or transition-metal hydroxides, such as, for example, Ca(OH)₂, Mg(OH)₂, Al(OH)₃, Zn(OH)₂. The mixing ratio determines the brightness and the yellow content of the coating. In addition, the color of the pigment can be adjusted by means of the amount of precipitated hydroxide mixture.
precipitation of iron-containing double hydroxides of the general formula [FeM²₂OH)₆]X. M² here can be =Mg^{II}, Ca^{II}, Sr^{II}, Ba^{II}, Zn^{II}. In this case, the stoichiometric ratio of Fe³⁺ and M² is prespecified. The color can then be adjusted by means of the amount of precipitated double hydroxide and the color of the M² cation.

In the three cases mentioned above, the final color of the pigment can additionally be influenced very specifically by the drying or calcination temperature.

Due to the content of iron ions or ions of other transition metals, there is of course a risk of the shelf life being adversely affected with respect to destruction of the cosmetic active substance and/or the organic formulation constituents. It is accordingly also of major advantage in this case to passivate these “skin corrector” pigments thermochemically and photochemically by means of an SiO₂ and/or Al₂O₃ layer. Passivation is the process of making a material inactive or inert. Aluminum, for example, is passivated when it reacts with the air to form a protective layer of aluminium oxide, which prevents further air-aluminum reaction. The commercially available metal flake pigments, for example from Silberline Ltd. or Eckart, are free-flowing powders which are offered in a slurry with a solvent like butyl glycol, ethanol or isopropyl alcohol.

In order to increase the light, water and weather stability, it is frequently advisable, depending on the area of application, to subject the finished effect pigment to post-coating or post-treatment. Suitable post-coatings or post-treatments are, for example, the processes described in German Patent 22 15 191, DE-A 31 51 354, DE-A 32 35 017 or DE-A 33 34 598. This post-coating further increases the chemical and photochemical stability or simplifies handling of the pigment, in particular incorporation into various media. In order to improve the wettability, dispersibility and/or compatibility with the user media, functional coatings of Al₂O₃ or ZrO₂ or mixtures thereof can, for example, be applied to the pigment surface. Furthermore, organic post-coatings are possible, for example with silanes, as described, for example, in EP 0 090259, EP 0 634 459, WO 99/57204, WO 96/32446, WO 99/57204, U.S. Pat. No. 5,759,255, U.S. Pat. No. 5,571,851, WO 01/92425 or in J. J. Ponjeé, Philips Technical Review, Vol. 44, No. 3, 81 ff., and P. H. Harding J. C. Berg, J. Adhesion Sci. Technol. Vol. 11 No. 4, pp. 471-493.

The choice of final post-coating is only limited inasmuch as it should be ensured that surface-reactive transition-metal compounds or transition-metal cations are not re-introduced into the end product in a disadvantageously large amount.

The pigments according to the invention having a mass tone are compatible with a multiplicity of color systems, preferably from the area of paints, coatings, printing inks and cosmetic formulations. For the preparation of printing inks, for example for gravure printing, flexographic printing, offset printing, offset overprint varnishing, a multiplicity of binders, in particular water-soluble types, is suitable, as marketed, for example, by BASF, Marabu, Pröll, Sericol, Hartmann, Gebr. Schmidt, Sicpa, Aarberg, Siegberg, GSB-Wahl, Folimann, Ruco or Coates Screen INKS GmbH. The printing inks can have a water-based or solvent-based structure. The pigments are furthermore also suitable for the laser marking of paper and plastics and for applications in the agricultural sector, for example for greenhouse sheeting, and, for example, for coloring tarpaulins.

As a consequence of their increased chemical stabilisation, the effect pigments according to the invention are particularly suitable for polymers, in particular plastics. The plastics preferably comprise 0.01-10% by weight, in particular 0.1-5% by weight and very particularly preferably 0.2-2.5% by weight, of the pigments according to the invention. Even at low pigment concentrations of <0.5% by weight, no significant dark yellowing of plastics comprising phenol-containing additives is evident. Furthermore, the effect pigments are suitable for coating formulations.

Since the effect pigments according to invention having naturally colored layers of transition-metal oxides, transition-metal hydroxyoxides and transition-metal hydroxides/hydroxide mixtures (for example comprising Fe/Ti, Fe, Cu) frequently exhibit a particularly “noble” hue (i.e., an elegant and expensive touch, luxurious appearance, combined with high quality) in the gold, brass, bronze, copper region and are generally distinguished by metallic luster and high brightness, they can be used to achieve particularly effective effects in the various application media, for example in cosmetic formulations, such as nail varnishes, lipsticks, compact powders, gels, lotions, emulsions, soaps, toothpastes and particularly preferably in self-tanning formulations.

The effect pigments according to the invention can, in a preferred type of application, be combined very well with cosmetic active compounds. Suitable active compounds are, for example, insect repellents, inorganic UV filters, such as, for example, TiO₂, UV A/BC protective filters (for example OMC, B3, MBC), also in encapsulated form, antiageing active compounds, vitamins and derivatives thereof (for example vitamin A, C, E, etc.), and further cosmetic active compounds, such as, for example, bisabolol, LPO, VTA, ectoin, emblica, allantoin, bioflavonoids and derivatives thereof.

In self-tanning creams, lotions, sprays, etc., comprising, for example, the self-tanning agents DHA (1,3-dihydroxyacetone) or erythrulose or a mixture of the two substances and an effect pigment having a final TiO₂ or Fe₂O₃ layer, the DHA is degraded relatively rapidly in the formulation in a period of 3 months, in particular if the TiO₂ is in the anatase modification. On use of the pigments according to the invention in the formulation, the action of the DHA is, by contrast, fully retained.

The self-tanning agent substances are usually sprayed onto the skin or applied manually as a solution or emulsion. Compared with the pigments from the prior art, the shelf life of self-tanning creams, lotions or sprays comprising the pigments according to the invention with the common self-tanning agent substances, such as, for example, 1,3-dihydroxyacetone and erythrulose, is significantly increased.

The self-tanning creams/sprays preferably comprise 0.5-5.0% by weight, preferably 1-3% by weight, of effect pigments according to the invention and the self-tanning substance in amounts of 0.01 to 20% by weight, preferably in amounts of 0.05% by weight to 10% by weight, in particular 1-8% by weight. In a mixture of self-tanning agent substances, the per cent by weight ratio is preferably between 1:10 and 10:1. A preferred mixture of self-tanning agent substances is the mixture of DHA and erythrulose. For example, DHA:erythrulose mixing ratios of 2:1 and 3:1 are employed. A further preferred self-tanning agent is so-called “DHA Plus”. Besides 1,3-dihydroxyacetone and magnesium stearate, this comprises sodium disulfite (Na₂S₂O₅, INCl:Sodium Disulfite) in order to mask/eliminate/neutralise the formaldehyde.

It goes without saying that, for the various applications, the effect pigments according to the invention can also advantageously be used as a blend with organic dyes, holographic pigments, LCPs (liquid crystal polymers), organic pigments or inorganic pigments, such as, for example, transparent and opaque white, colored and black pigments, and with flake-form iron oxides, and conventional transparent, colored and black luster pigments based on metal-oxide-coated mica, glass, Al₂O₃, Fe₂O₃ and SiO₂ and passivated aluminium flakes, etc. The pigments according to the invention can be mixed in any ratio with commercially available pigments and fillers.

Examples of fillers which may be mentioned are natural and synthetic mica, nylon powder, pure or filled melamine resins, talc, SiO₂, glasses, kaolin, oxides or hydroxides of aluminium, magnesium, calcium, zinc, BiOCl, barium sulfate, calcium sulfate, calcium carbonate, magnesium carbonate, carbon, and physical or chemical combinations of these substances.

There are no restrictions regarding the particle shape of the filler. It can be, for example, flake-form, spherical or needle-shaped, but also completely irregularly shaped, in accordance with requirements.

Cosmetic formulations which comprise the effect pigments according to the invention can belong to the lipophilic, hydrophilic or hydrophobic type. In the case of heterogeneous formulations having discrete aqueous and non-aqueous phases, the effect pigments according to the invention may in each case be present in only one of the two phases or also distributed over both phases.

The pH values of the formulations can be between 1 and 14, preferably between 2 and 11 and particularly preferably between 5 and 8.

The concentrations of the effect pigments according to the invention in the formulation are not limited. They can be—depending on the application—between 0.001 (rinse-off products, for example shower gels)—100% (for example luster-effect articles for particular applications).

The pigments according to the invention can of course also be combined in the formulations with any type of raw materials and assistants. These include, inter alia, oils, fats, waxes, film formers, preservatives, surfactants, antioxidants, such as, for example, vitamin C or vitamin E, stabilizers, odor enhancers, silicone oils, emulsifiers, solvents, such as, for example, ethanol, or ethyl acetate or butyl acetate, and assistants which generally determine the applicational properties, such as, for example, thickeners and rheological additives, such as, for example, bentonites, hectorites, silicon dioxides, Ca silicates, gelatines, high-molecular-weight carbohydrates and/or surface-active assistants, etc.

Organic UV filters are generally incorporated into cosmetic formulations in an amount of 0.5-10% by weight, preferably 1-8% by weight, inorganic filters in an amount of 0.1-30% by weight.

The compositions according to the invention may, in addition, comprise further conventional skin-protecting or skin-care active compounds. These may in principle be all active compounds known to the person skilled in the art. Particularly preferred active compounds are pyrimidinecarboxylic acids and/or aryl oximes.

Of the cosmetic applications, particular mention may be made of the use of ectoin and ectoin derivatives for the care of aged, dry or irritated skin. Thus, EP-A-0 671 161 discloses that ectoin and hydroxyectoin can be employed in cosmetic compositions, such as, for example, powders, soaps, surfactant-containing cleansing products, lipsticks, rouge, make-up, care creams and sunscreen preparations.

Application forms of the cosmetic formulations which may be mentioned are, for example: solutions, suspensions, emulsions, PIT emulsions, pastes, ointments, gels, creams, lotions, powders, soaps, surfactant-containing cleansing preparations, oils, aerosols and sprays. Examples of other application forms are sticks, shampoos and shower preparations. Any desired customary vehicles, assistants and, if desired, further active compounds may be added to the composition.

Ointments, pastes, creams and gels may comprise the customary vehicles, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary propellants, for example chlorofluorocarbons, propaneabutane or dimethyl ether.

Solutions and emulsions may comprise the customary vehicles, such as, for example, solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary vehicles, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as, for example, fatty acid esters, fatty alcohols, silicone oils, natural oils, such as vegetable oils and oily plant extracts, paraffin oils, lanolin oils, or mixtures of these substances.

The cosmetic compositions may exist in various forms. Thus, they can be, for example, a solution, a water-free composition, an emulsion or microemulsion of the water-in-oil (W/O) or oil-in-water (O/W) type, a multiple emulsion, for example of the water-in-oil-in-water (W/O/W) type, a gel, a solid stick, an ointment or an aerosol. It is also advantageous to administer ectoins in encapsulated form, for example in collagen matrices and other conventional encapsulation materials, for example as cellulose encapsulations, in gelatine, wax matrices or liposomally encapsulated. In particular, wax matrices, as described, for example, in DE-A 43 08 282, have proven favourable. Preference is given to emulsions. O/W emulsions are particularly preferred. Emulsions, W/O emulsions and O/W emulsions are obtainable in a conventional manner.

Further embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

Solid sticks consist of natural or synthetic waxes and oils, fatty alcohols, fatty acids, fatty acid esters, lanolin and other fatty substances.

If a composition is formulated as an aerosol, the customary propellants, such as alkanes, fluoroalkanes and chlorofluoroalkanes, are generally used.

The cosmetic composition may also be used to protect the hair against photo-chemical damage in order to prevent color changes, bleaching or damage of a mechanical nature. In this case, a suitable formulation is in the form of a rinse-out shampoo, lotion, gel or emulsion, the composition in question being applied before or after shampooing, before or after coloring or bleaching or before or after permanent waving. It is also possible to select a composition in the form of a lotion or gel for styling or treating the hair, in the form of a lotion or gel for brushing or blow-waving, in the form of a hair lacquer, permanent waving composition, colorant or bleach for the hair. The composition having light-protection properties may comprise adjuvants, such as surfactants, thickeners, polymers, softeners, preservatives, foam stabilizers, electrolytes, organic solvents, silicone derivatives, oils, waxes, antigrease agents, dyes and/or pigments which color the composition itself or the hair, or other ingredients usually used for hair care.

The invention thus also relates to the use of the effect pigments in formulations, such as paints, coatings, automobile paints, powder coatings, printing inks, security printing inks, plastics, ceramic materials, glasses, paper, in paper coating, in toners for electrophotographic printing processes, in seed, in greenhouse sheeting and tarpaulins, as absorbers in the laser marking of paper and plastics, as absorbers in the laser welding of plastics, cosmetic formulations, for the preparation of pigment pastes with water, organic and/or aqueous solvents, for the preparation of pigment compositions and dry preparations, such as, for example, granules.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

### EXAMPLES

### I. Preparation of the Pigments

### Example 1

### Natural mica+TiO₂+FeOOH+SiO₂

200 g of natural potassium mica of the 10-60 μm fraction are made up to a concentration of 100 g/l with deionized water. The suspension is warmed to 80° C. The pH is subsequently adjusted to 1.8 using hydrochloric acid (15% HCl). About 30 ml of a TiOCl₂ solution (400 g/l) are metered in at a constant pH while counter-titrating with 32% w/w NaOH. When the metered addition is complete, the mixture is stirred for a further 10 min. A pH of 4.0 is subsequently set using NaOH. An FeOOH layer is then precipitated on to a gold-green color at constant pH by metered addition of an FeCl₃ solution (14.25% of Fe), and the mixture is stirred for a further 30 min.

The pH is subsequently adjusted to pH 9.0 using dilute sodium hydroxide solution. 100 ml of a sodium water-glass solution having a silicic acid content of 5% are added over the course of 2 hours, during which the pH is kept constant by means of 2.5% sulfuric acid. The mixture is subsequently stirred for a further 30 minutes, and the pH is then adjusted to 7.5 over the course of 30 minutes using sulfuric acid. After a post-stirring time of 30 minutes, the post-coated pigment is separated off from the supernatant reaction solution by filtration and washed until salt-free. The pigment is dried at 110° C. and sieved in accordance with the desired particle size.

The pigment according to the invention obtained in this way exhibits pale-green interference with gold-ochre mass tone. It contains a coating of silicon dioxide having a layer thickness of about 20-35 nm.

### Example 2

### Synthetic mica+Fe₂O₃+5% of SiO₂/2.5% of Al₂O₃ (2:1)

75 g of synthetic mica flakes having a particle size of 10-50 μm are heated to 75° C. with stirring in 1.5 I of deionized water.

The pH of the suspension is then adjusted to 3.0 using 5% hydrochloric acid. A 3% by weight (based on the Fe³⁺ content) iron chloride solution is subsequently metered in, during which the pH is kept constant by simultaneous dropwise addition of 32% sodium hydroxide solution. The color properties are monitored during preparation of the pigment by color measurement during the process, and the precipitation process is controlled in accordance with the hue (hue angle arc tan b*/a*). With increasing layer thickness of the iron hydroxyoxide precipitated in this way, bronze-colored, orange-red, red-brown to red-violet hues are passed through.

When the desired end point has been reached, the mixture is stirred for a further 15 minutes.

The pigment suspension obtained is adjusted to pH 9 using 32% NaOH. After addition of the sodium water-glass solution having a silicic acid content of 5% (diluted with water in the ratio 1:1; 39 ml in 1000 ml of water) over the course of 2 h, the mixture is stirred for 15 minutes, and the pH is adjusted to pH 6.5 using conc. HCl. After the mixture has been stirred for 15 minutes, 5.33 g of AlCl₃×6 H₂O and 3.20 g of sodium sulfate in solid form are added. The suspension obtained is stirred at 90° C. for 1 h. The aqueous solution is subsequently filtered with suction, washed until chloride-free, and the product is dried. The product is calcined at 700° C. for 30 minutes.

The pigment according to the invention obtained in this way contains a coating of silicon oxide/aluminium oxide having a layer thickness of about 30 nm.

### Example 3

### Natural mica+Fe₂O₃+SiO₂ (doped with K⁺)

75 g of mica flakes (natural) having a particle size of 10-50 μm are heated to 75° C. with stirring in 1.5 I of deionized water. The pH of the suspension is then adjusted to 3.0 using conc. hydrochloric acid.

A 5% by weight (based on the Fe³⁺ content) iron chloride solution is then metered in, during which the pH is kept constant by simultaneous dropwise addition of 32% sodium hydroxide solution. The color properties are monitored during preparation of the pigment by color measurement during the process, and the precipitation process is controlled in accordance with the hue (hue angle arc tan b*/a*). With increasing layer thickness of the precipitated iron hydroxyoxide, bronze-colored, orange-red, red-brown to red-violet hues are passed through.

When the desired end point has been reached, the mixture is stirred for a further 15 minutes.

The pH is adjusted to pH 9.0 using dilute potassium hydroxide solution. 100 ml of a potassium water-glass solution having a silicic acid content of 5% are added over the course of 2 hours, during which the pH is kept constant by means of 2.5% sulfuric acid. The mixture is subsequently stirred for a further 30 minutes, and the pH is then adjusted to 7.5 over the course of 30 minutes using sulfuric acid. After a post-stirring time of 30 minutes, the post-coated pigment is separated off from the supernatant reaction solution by filtration and washed. After drying at 100 to 150° C., the pigment is calcined at 700° C. for 45 minutes.

The pigment obtained in this way contains a coating of silicon dioxide having a layer thickness of about 20-30 nm.

### Example 4

### Natural mica+Fe₂O₃+SiO₂ (Variation in the Amount of SiO₂, Based on the Base Pigment)

### Example 4a

### Natural mica +Fe₂O₃ (Comparison)

The coating of mica with Fe₂O₃ is carried out analogously to Example 3 to the desired color end point in the bronze region. The mixture is stirred for a further 30 minutes, and the coated substrate is subsequently separated off from the supernatant by filtration and washed. The pigment precursor is subsequently dried at 150° C. and calcined at 700° C. for 45 minutes.

The resultant pigment serves as comparison without post-coating and is employed directly for further post-coatings for Examples 4b and 4c.

### Example 4b

### Bronze-Colored Fe₂O₃ Pigment According to Example 4a+15% of SiO₂

200 g of calcined, bronze-colored pigment having a particle size of 10-60 μm, prepared analogously to Example 4a, are resuspended in 2.4 I of deionized water and heated to 70° C. with stirring. The pH is adjusted to pH 9.0 using dilute sodium hydroxide solution.

383 g of a sodium water-glass solution having an SiO₂ content of 8% are metered in over the course of 3 hours, during which the pH is kept constant by means of 10% hydrochloric acid. The mixture is subsequently stirred for a further 30 minutes, and the pH is then adjusted to 5 over the course of 30 minutes using hydrochloric acid. After a post-stirring time of 30 minutes, the post-coated pigment is separated off from the supernatant reaction solution by filtration and washed until salt-free. The pigment is dried at 150° C. and calcined at 700° C. for 45 minutes.

The pigment according to the invention obtained in this way is bronze-colored.

### Example 4c

### Bronze-colored Fe₂O₃ pigment according to Example 4a+30% of SiO₂

200 g of calcined, bronze-colored pigment having a particle size of 10-60 μm, prepared analogously to Example 4a, are resuspended in 2.4 I of deionized water and heated to 70° C. with stirring. The pH is adjusted to pH 9.0 using dilute sodium hydroxide solution.

766 g of a sodium water-glass solution having an SiO₂ content of 8% are metered in over the course of 7 hours, during which the pH is kept constant by means of 10% hydrochloric acid. The mixture is subsequently stirred for a further 30 minutes, and the pH is then adjusted to 5 over the course of 30 minutes using hydrochloric acid. After a post-stirring time of 30 minutes, the post-coated pigment is separated off from the supernatant reaction solution by filtration and washed until salt-free. The pigment is dried at 150° C. and calcined at 700° C. for 45 minutes.

The pigment according to the invention obtained in this way is bronze-colored.

### II. Quantitative Determination of the Pigment Reactivity to DHA in Self-Tanning Formulations

For comparative experiments, 5% of dihydroxyacetone and 5% of pigment from Examples 4, 4a and 4b are in each case incorporated into an O/W cream and into an aqueous solution. (Comparison: only with dihydroxyacetone)

Cream formulation (phase A):

| Raw materials | INCI | Wt. [%] |
|---|---|---|
| Tego Care 150 | Glyceryl Stearate, Steareth-26, Ceteth- | 8.00 |
| | 20, Stearyl Alcohol | |
| Lanette O | Cetearyl Alcohol | 1.50 |
| Tegosoft Liquid | Cetearyl Ethylhexanoate | 5.00 |
| Miglyol 812 N | Caprylic/Capric Triglyceride | 5.00 |
| Abil-Wax 2434 | Stearoxy Dimethicone | 1.00 |
| Dow Corning 200 | Dimethicone | 0.50 |
| (100 cs) | | |
| Propyl 4-hydroxy- | Propylparaben | 0.05 |
| benzoate | | |

Cream formulation (phase B):

| Raw materials | INCI | Wt. [%] |
|---|---|---|
| Pigment from | | 5.00 |
| Example 4, 4a or | | |
| 4b | | |
| 1,2-Propanediol | Propylene glycol | 3.00 |
| Methyl 4-hydroxy- | Methlyparaben | 0.15 |
| benzoate | | |
| Water, demineralized | Aqua (Water) | 55.80 |

Cream formulation (phase C):

| Raw materials | INCI | Wt. [%] |
|---|---|---|
| Dihydroxyacetone | Dihydrocyacetone | 5.00 |
| Water, demineralized | Aqua (Water) | 10.00 |

Preparation:

Heat phase A and B to 75-80° C. Rapidly add phase B to phase A. Homogenise.

Cool with stirring and add phase C at 40° C.
The samples are stored for 3 months at room temperature and at 40° C. with exclusion of light.
Enzymatic DHA determination is carried out after preparation (initial value) and after 3 months
Requirement for stable DHA formulations:DHA degradation at most 20%

Result:

| | Initial | After 3 months | Degradation of |
|---|---|---|---|
| Samples | values | at 40° C. | DHA in % |
| Pigment from Ex. 4a (with | 5.0 | 1.6 | 68.0 |
| 0% of SiO₂), + 5% of DHA | | | |
| Pigment from Ex. 4b (with | 4.9 | 4.3 | 12.2 |
| 15% of SiO₂), + 5% of | | | |
| DHA | | | |
| Pigment from Ex. 4c (with | 5.1 | 5.1 | 0 |
| 30% of SiO₂), + 5% of | | | |
| DHA | | | |
| 5% of DHA | 4.8 | 4.3 | 10.4 |

### III. Use Examples:

### Use Example 1

### Care Self-Tanning Cream (O/W)

| Ingredients | | INCI | [%] |
|---|---|---|---|
| Phase A | | | |
| Montanov 68 | (1) | Cetearyl Alcohol, Cetearyl | 4.00 |
| | | Glucoside | |
| Span 60 | (2) | Sorbitan Stearate | 1.50 |
| Lanette O | (3) | Cetearyl Alcohol | 1.00 |
| Cosmacol ELI | (4) | C12-13 Alkyl Lactate | 3.00 |
| Cosmacol EMI | (4) | DI-C12-13 Alkyl Malate | 1.50 |
| Arlamol HD | (2) | Isohexadecane | 3.00 |
| Dow Corning 9040 silicone | (5) | Cyclomethicone, Dimethicone | 1.00 |
| elastomer blend | | Crosspolymer | |
| RonaCare ® tocopherol | (6) | Tocopheryl Acetate | 0.50 |
| acetate | | | |
| Propyl 4-hydroxybenzoate | (6) | Propylparaben | 0.05 |
| Phase B | | | |
| RonaCare ® ectoin | (6) | Ectoin | 0.50 |
| Pigment from Example 4b | | | 2.00 |
| Glycerol, anhydrous | (6) | Glycerin | 2.00 |
| FD&C Yellow No. 6 W082 | (8) | CI 15985 | 0.01 |
| Methyl 4-hydroxybenzoate | (6) | Methylparaben | 0.15 |
| Water, demineralized | | Aqua (Water) | 64.09 |
| Phase C | | | |
| Sepigel 305 | (1) | Laureth-7, Polyacrylamide, | 0.50 |
| | | C13-14 Isoparaffin | |
| Phase D | | | |
| Dihydroxyacetone | (6) | Dihydroxyacetone | 5.00 |
| Water, demineralized | | Aqua (Water) | 10.00 |
| Phase E | | | |
| Babylon fragrance | (9) | Perfume | 0.20 |

Preparation:

Phase A and B are warmed separately to 75° C. Phase B is then slowly admixed with phase A with stirring. Phase C is admixed with phase A/B at 60° C. using a hand stirrer and homogenised. Allow to cool to 40° C. and stir in phase D and phase E.

Notes:

pH_{23° C.}=4.0

Viscosity: 18,600 cps (Brookfield model RVT DV-II, Helipath spindle C, 10 rpm) at 23° C.

Sources of Supply

(1) Seppic

(2) Uniqema

(3) Cognis GmbH

(4) Condea Chimica D.A.C. S.p.A.

(5) Dow Corning

(6) Merck KGaA/Rona®

(7) D. D. Williamson

(8) Les Colorants Wackherr SA

(9) Drom

### Use Example 2

### Self-Tanning Cream (O/W)

| Ingredients | | INCI | [%] |
|---|---|---|---|
| Phase A | | | |
| Tego Care 150 | (1) | Glyceryl Stearate, | 8.00 |
| | | Steareth-26, Ceteth-20, | |
| | | Stearyl Alcohol | |
| Paraffin liquid | (2) | Parffinum Liquidum | 12.00 |
| | | (Mineral Oil) | |
| Paraffin pourable | (2) | Paraffin | 2.00 |
| Miglyol 812 N | (3) | Caprylic/Capric Triglyceride | 3.00 |
| Isopropyl myristate | (4) | Isopropyl Myristate | 2.00 |
| Propyl 4-hydroxybenzoate | (2) | Propylparaben | 0.15 |
| Phase B | | | |
| 1,2-Propanediol | (2) | Propylene Glycol | 4.00 |
| Sorbitol F liquid | (2) | Sorbitol | 2.00 |
| Water, demineralized | | Aqua (Water) | 47.40 |
| Methyl 4-hydroxybenzoate | (2) | Methylparaben | 0.15 |
| Pigment from Example 1 | | | 2.00 |
| Phase C | | | |
| Dihydroxyacetone | (2) | Dihydroxyacetone | 5.00 |
| Water, demineralized | | Aqua (Water) | 11.80 |
| Phase D | | | |
| Fragrance (q.s.) | | Perfume | 0.50 |

Preparation:

Phase A is warmed to 80° C. and phase B to 75° C. Phase A is then slowly added to phase B with stirring. The mixture is homogenised for one minute at 65° C. using a hand stirrer. Allow to cool to 40° C. and stir in phase C. Cool further to 35° C. and mix in phase D with stirring.

Notes:

pH_{23° C.}=4.6

Viscosity: 42,500 mPas (Brookfield RVT, sp. C, 10 rpm) at 23° C.

Sources of Supply:

(1) Degussa-Goldschmidt AG

(2) Merck KGaA/Rona®

(3) Sasol Germany GmbH

(4) Cognis GmbH

### Use Examples 3-14

### Self-Tanning Cream (O/W)

| Ingredients | INCI | 3 | 4 | 5 | 6 | 7 | 8 | 9 | 10 | 11 | 12 | 13 | 14 |
|---|---|---|---|---|---|---|---|---|---|---|---|---|---|
| Phase A | | | | | | | | | | | | | |
| Tego Care 150 | Glyceryl | 8.00 | 8.00 | 8.00 | 8.00 | 8.00 | 8.00 | 8.00 | 8.00 | 8.00 | 8.00 | 8.00 | 8.00 |
| | Stearate, | | | | | | | | | | | | |
| | Steareth-26, | | | | | | | | | | | | |
| | Ceteth-20, | | | | | | | | | | | | |
| | Stearyl | | | | | | | | | | | | |
| | Alcohol | | | | | | | | | | | | |
| Lanette O | Cetearyl | 1.50 | 1.50 | 1.50 | 1.50 | 1.50 | 1.50 | 1.50 | 1.50 | 1.50 | 1.50 | 1.50 | 1.50 |
| | Alcohol | | | | | | | | | | | | |
| Tegosoft Liquid | Cetearyl | 6.50 | 5.50 | 5.50 | 5.50 | 5.50 | 5.50 | 5.50 | 6.50 | 5.50 | 5.50 | 5.50 | 5.50 |
| | Ethylhexanoate | | | | | | | | | | | | |
| Miglyol 812 N | Caprylic/- | 6.50 | 5.50 | 5.50 | 5.50 | 5.50 | 5.50 | 5.50 | 6.50 | 5.50 | 5.50 | 5.50 | 5.50 |
| | Capric | | | | | | | | | | | | |
| | Triglyceride | | | | | | | | | | | | |
| Abil-Wax 2434 | Stearoxy | 1.20 | 1.00 | 1.00 | 1.00 | 1.00 | 1.00 | 1.00 | 1.20 | 1.00 | 1.00 | 1.00 | 1.00 |
| | Dimethicone | | | | | | | | | | | | |
| Dow Corning 200 | Dimethicone | 0.50 | 0.50 | 0.50 | 0.50 | 0.50 | 0.50 | 0.50 | 0.50 | 0.50 | 0.50 | 0.50 | 0.50 |
| (100cs) | | | | | | | | | | | | | |
| RonaCare ® | Tocopheryl | | 0.50 | 0.50 | 0.50 | 0.50 | 0.50 | 0.50 | | 0.50 | 0.50 | 0.50 | 0.50 |
| tocopherol acetate | Acetate | | | | | | | | | | | | |
| Propyl 4- | Propyl- | 0.05 | 0.05 | 0.05 | 0.05 | 0.05 | 0.05 | 0.05 | 0.05 | 0.05 | 0.05 | 0.05 | 0.05 |
| hydroxybenzoate | paraben | | | | | | | | | | | | |
| Phase B | | | | | | | | | | | | | |
| RonaCare ® ectoin | Ectoin | 0.30 | 0.30 | 0.30 | 0.30 | 0.30 | 0.30 | 0.30 | 0.30 | 0.30 | 0.30 | 0.30 | 0.30 |
| 1,2-Propanediol | Propylene | 3.00 | 3.00 | 3.00 | 3.00 | 3.00 | 3.00 | 3.00 | 3.00 | 3.00 | 3.00 | 3.00 | 3.00 |
| | Glycol | | | | | | | | | | | | |
| Methyl 4- | Methyl- | 0.15 | 0.15 | 0.15 | 0.15 | 0.15 | 0.15 | 0.15 | 0.15 | 0.15 | 0.15 | 0.15 | 0.15 |
| hydroxybenzoate | paraben | | | | | | | | | | | | |
| Water, | Aqua (Water) | 60.20 | 59.90 | 53.90 | 61.40 | 58.90 | 51.40 | 61.40 | 57.20 | 51.40 | 60.90 | 57.90 | 48.90 |
| demineralized | | | | | | | | | | | | | |
| Phase C | | | | | | | | | | | | | |
| Dihydroxyacetone | Dihydroxy- | 1.00 | 2.00 | 5.00 | 1.00 | 2.00 | 5.00 | 1.00 | 2.00 | 5.00 | 1.00 | 2.00 | 5.00 |
| | acetone | | | | | | | | | | | | |
| Trihydroxyethyl- | Troxerutin | | | | 0.50 | 1.00 | 2.50 | | | | 0.50 | 1.00 | 2.50 |
| rutine | | | | | | | | | | | | | |
| Pigment from | | 1.00 | 2.00 | 5.00 | 1.00 | 2.00 | 5.00 | 1.00 | 2.00 | 5.00 | 1.00 | 2.00 | 5.00 |
| Example 2 | | | | | | | | | | | | | |
| Erythrulose | Erythrulose | | | | | | | 0.50 | 1.00 | 2.50 | 0.50 | 1.00 | 2.50 |
| Water, | Aqua (Water) | 10.00 | 10.00 | 10.00 | 10.00 | 10.00 | 10.00 | 10.00 | 10.00 | 10.00 | 10.00 | 10.00 | 10.00 |
| demineralized | | | | | | | | | | | | | |
| Phase D | | | | | | | | | | | | | |
| Sunshine fragrance | Perfume | 0.10 | 0.10 | 0.10 | 0.10 | 0.10 | 0.10 | 0.10 | 0.10 | 0.10 | 0.10 | 0.10 | 0.10 |

Preparation:

Phase A is warmed to 80° C. and phase B is warmed to 80° C. Phase B is then slowly added to phase A with stirring. The mixture is homogenised. Allow to cool to 40° C. and stir in phase C. Mix in phase D with stirring.

### Use Examples 15-26

### Self-Tanning Lotion (W/O)

| Ingredients | INCI | 15 | 16 | 17 | 18 | 19 | 20 | 21 | 22 | 23 | 24 | 25 | 26 |
|---|---|---|---|---|---|---|---|---|---|---|---|---|---|
| Phase A | | | | | | | | | | | | | |
| Abil EM 97 | Bis-PEG/PPG-14/14, | 1.50 | 1.50 | 1.50 | 1.50 | 1.50 | 1.50 | 1.50 | 1.50 | 1.50 | 1.50 | 1.50 | 1.50 |
| | Dimethicone, | | | | | | | | | | | | |
| | Cyclopentasiloxane, | | | | | | | | | | | | |
| | Cetyl PEG/PPG-10/1 | | | | | | | | | | | | |
| Abil EM 90 | Dimethicone | 1.30 | 1.30 | 1.30 | 1.30 | 1.30 | 1.30 | 1.30 | 1.30 | 1.30 | 1.30 | 1.30 | 1.30 |
| Dow Corning 345 | Cyclomethicone | 13.00 | 13.00 | 13.00 | 13.00 | 13.00 | 13.00 | 13.00 | 13.00 | 13.00 | 13.00 | 13.00 | 13.00 |
| Ceraphyl 368 | Ethylhexyl Palmitate | 2.00 | 2.00 | 2.00 | 2.00 | 2.00 | 2.00 | 2.00 | 2.00 | 2.00 | 2.00 | 2.00 | 2.00 |
| SFE 839 | Cyclopentasiloxane, | 5.00 | 5.00 | 5.00 | 5.00 | 5.00 | 5.00 | 5.00 | 5.00 | 5.00 | 5.00 | 5.00 | 5.00 |
| | Dimethicone/- | | | | | | | | | | | | |
| | Vinyldimethicone | | | | | | | | | | | | |
| | Crosspolymer | | | | | | | | | | | | |
| Tegosoft DEC | Diethylhexyl Carbonate | 5.00 | 5.00 | 5.00 | 5.00 | 5.00 | 5.00 | 5.00 | 5.00 | 5.00 | 5.00 | 5.00 | 5.00 |
| Babylon fragrance | Perfume | 0.30 | 0.30 | 0.30 | 0.30 | 0.30 | 0.30 | 0.30 | 0.30 | 0.30 | 0.30 | 0.30 | 0.30 |
| Phase B | | | | | | | | | | | | | |
| Dihydroxyacetone | Dihydroxyacetone | 1.00 | 2.00 | 5.00 | 1.00 | 2.00 | 5.00 | 1.00 | 2.00 | 5.00 | 1.00 | 2.00 | 5.00 |
| Trihydroxyethylrutin | Troxerutin | | | | 0.50 | 1.00 | 2.50 | 2.00 | | | | | |
| Pigment according to | | 1.00 | 2.00 | 5.00 | 1.00 | 2.00 | 5.00 | 1.00 | 2.00 | 5.00 | 1.00 | 2.00 | 5.00 |
| Example 3 | | | | | | | | | | | | | |
| Erythrulose | Erythrulose | | | | | | | 0.50 | 1.00 | 2.50 | 0.50 | 1.00 | 2.50 |
| RonaCare ® ectoin | Ectoin | 0.30 | 0.30 | 0.30 | 0.30 | 0.30 | 0.30 | 0.30 | 0.30 | 0.30 | 0.30 | 0.30 | 0.30 |
| 1,2-Propanediol | Propylene Glycol | 18.00 | 18.00 | 18.00 | 18.00 | 18.00 | 18.00 | 18.00 | 18.00 | 17.00 | 17.00 | 17.00 | 17.00 |
| Glycerol, anhydrous | Glycerin | 3.00 | 3.00 | 3.00 | 3.00 | 3.00 | 3.00 | 3.00 | 3.00 | 3.00 | 3.00 | 3.00 | 3.00 |
| Magnesium sulfate | Magnesium Sulfate | 2.00 | 2.00 | 2.00 | 2.00 | 2.00 | 2.00 | 2.00 | 2.00 | 2.00 | 2.00 | 2.00 | 2.00 |
| heptahydrate | | | | | | | | | | | | | |
| Ethanol 96% | Alcohol | 8.00 | 8.00 | 8.00 | 8.00 | 8.00 | 8.00 | 8.00 | 8.00 | 8.00 | 8.00 | 8.00 | 8.00 |
| Phenonip | Phenoxyethanol, | 1.00 | 1.00 | 1.00 | 1.00 | 1.00 | 1.00 | 1.00 | 1.00 | 1.00 | 1.00 | 1.00 | 1.00 |
| | Butylparaben, | | | | | | | | | | | | |
| | Ethylparaben, | | | | | | | | | | | | |
| | Methylparaben | | | | | | | | | | | | |
| Water, demineralized | Aqua (Water) | 37.60 | 35.60 | 29.60 | 37.10 | 34.60 | 27.10 | 37.10 | 34.60 | 28.10 | 37.60 | 34.60 | 25.60 |

Preparation:

Dissolve magnesium sulfate heptahydrate in the water of phase B. Addition of the remaining ingredients of phase B. Slowly add phase B to phase A with stirring using a hand mixer. Stir rapidly for 2 minutes and homogenise for 2 minutes.

### Use Examples 27-38

### Hydrogel

| Ingredients | INCI | 27 | 28 | 29 | 30 | 31 | 32 | 33 | 34 | 35 | 36 | 37 | 38 |
|---|---|---|---|---|---|---|---|---|---|---|---|---|---|
| Phase A | | | | | | | | | | | | | |
| Dihydroxyacetone | Dihydroxyacetone | 1.00 | 2.00 | 5.00 | 1.00 | 2.00 | 5.00 | 1.00 | 2.00 | 5.00 | 1.00 | 2.00 | 5.00 |
| Trihydroxyethylrutine | Troxerutin | | | | 0.50 | 1.00 | 2.50 | | | | 0.50 | 1.00 | 2.50 |
| Pigment according to | | 1.00 | 2.00 | 5.00 | 1.00 | 2.00 | 5.00 | 1.00 | 2.00 | 5.00 | 1.00 | 2.00 | 5.00 |
| Example 4b | | | | | | | | | | | | | |
| Erythrulose | Erythrulose | | | | | | | 0.50 | 1.00 | 2.50 | 0.50 | 1.00 | 2.50 |
| RonaCare ® ectoin | Ectoin | 0.30 | 0.30 | 0.30 | 0.30 | 0.30 | 0.30 | 0.30 | 0.30 | 0.30 | 0.30 | 0.30 | 0.30 |
| 1,2-Propanediol | Propylene Glycol | 2.50 | 2.50 | 2.50 | 2.50 | 2.50 | 2.50 | 2.50 | 2.50 | 2.50 | 2.50 | 2.50 | 2.50 |
| Sorbitol F liquid | Sorbitol | 2.50 | 2.50 | 2.50 | 2.50 | 2.50 | 2.50 | 2.50 | 2.50 | 2.50 | 2.50 | 2.50 | 2.50 |
| Methyl 4- | Methylparaben | 0.20 | 0.20 | 0.20 | 0.20 | 0.20 | 0.20 | 0.20 | 0.20 | 0.20 | 0.20 | 0.20 | 0.20 |
| hydroxybenzoate | | | | | | | | | | | | | |
| Water, demineralized | Aqua (Water) | 31.00 | 29.00 | 23.00 | 30.50 | 28.00 | 20.50 | 30.50 | 28.00 | 20.50 | 30.00 | 27.00 | 18.00 |
| Phase B | | | | | | | | | | | | | |
| Water, demineralized | Aqua (Water) | 60.00 | 60.00 | 60.00 | 60.00 | 60.00 | 60.00 | 60.00 | 60.00 | 60.00 | 60.00 | 60.00 | 60.00 |
| Natrosol 250 HHR | Hydroxyethyl- | 1.50 | 1.50 | 1.50 | 1.50 | 1.50 | 1.50 | 1.50 | 1.50 | 1.50 | 1.50 | 1.50 | 1.50 |
| | cellulose | | | | | | | | | | | | |

Preparation:

Add Natrosol to phase B and homogenise.

### Use Examples 39-46

### Shower Lotion

| Ingredients | INCI | 39 | 40 | 41 | 42 | 43 | 44 | 45 | 46 |
|---|---|---|---|---|---|---|---|---|---|
| Phase A | | | | | | | | | |
| Dihydroxyacetone | Dihydroxyacetone | 8.00 | 10.00 | 8.00 | 10.00 | 8.00 | 10.00 | 8.00 | 10.00 |
| Trihydroxyethylrutine | Troxerutin | | | 2.00 | 3.00 | 2.00 | 3.00 | | |
| Pigment according to | | 3.00 | 5.00 | 3.00 | 5.00 | 3.00 | 5.00 | 3.00 | 5.00 |
| Example 1 | | | | | | | | | |
| Erythrulose | Erythrulose | | | | | 3.00 | 5.00 | 3.00 | 5.00 |
| RonaCare ® ectoin | Ectoin | 0.50 | 0.50 | 0.50 | 0.50 | 0.50 | 0.50 | 0.50 | 0.50 |
| 1,2-Propanediol | Propylene Glycol | 4.50 | 4.50 | 4.50 | 4.50 | 4.50 | 4.50 | 4.50 | 4.50 |
| Glycerol, anhydrous | Glycerin | 4.00 | 4.00 | 4.00 | 4.00 | 4.00 | 4.00 | 4.00 | 4.00 |
| Transcutol CG | Ethoxydiglycol | 3.00 | 3.00 | 3.00 | 3.00 | 3.00 | 3.00 | 3.00 | 3.00 |
| Arlasolve DMI | Dimethyl Isosorbide | 2.00 | 2.00 | 2.00 | 2.00 | 2.00 | 2.00 | 2.00 | 2.00 |
| Tween 80 | Polysorbate 80 | 0.50 | 0.50 | 0.50 | 0.50 | 0.50 | 0.50 | 0.50 | 0.50 |
| Walnut extract | Glycol | 1.50 | 1.50 | 1.50 | 1.50 | 1.50 | 1.50 | 1.50 | 1.50 |
| Caramel 050 | Caramel | 0.10 | 0.10 | 0.10 | 0.10 | 0.10 | 0.10 | 0.10 | 0.10 |
| Sunshine fragrance | Perfume | 0.30 | 0.30 | 0.30 | 0.30 | 0.30 | 0.30 | 0.30 | 0.30 |
| Phenonip | Phenoxyethanol, Butyl- | 0.80 | 0.80 | 0.80 | 0.80 | 0.80 | 0.80 | 0.80 | 0.80 |
| | paraben, Ethylparaben, | | | | | | | | |
| | Propylparaben, | | | | | | | | |
| | Methylparaben | | | | | | | | |
| Water, demineralized | Aqua (Water) | 73.00 | 69.80 | 69.80 | 71.00 | 66.80 | 70.80 | 71.80 | 71.80 |

Preparation:

Homogenise all ingredients with stirring.

### Use Examples 47-54

### Shower Lotion

| Ingredients | INCI | 47 | 48 | 49 | 50 | 51 | 52 | 53 | 54 |
|---|---|---|---|---|---|---|---|---|---|
| Phase A | | | | | | | | | |
| Dihydroxyacetone | Dihydroxyacetone | 8.00 | 10.00 | 8.00 | 10.00 | 8.00 | 10.00 | 8.00 | 10.00 |
| Trihydroxyethylrutine | Troxerutin | | | 2.00 | 3.00 | 2.00 | 3.00 | | |
| Pigment according to | | 3.00 | 5.00 | 3.00 | 5.00 | 3.00 | 5.00 | 3.00 | 5.00 |
| Example 1 | | | | | | | | | |
| Erythrulose | Erythrulose | | | | | 3.00 | 5.00 | 3.00 | 5.00 |
| RonaCare ® ectoin | Ectoin | 0.50 | 0.50 | 0.50 | 0.50 | 0.50 | 0.50 | 0.50 | 0.50 |
| 1,2-Propanediol | Propylene Glycol | 6.00 | 6.00 | 6.00 | 6.00 | 6.00 | 6.00 | 6.00 | 6.00 |
| Transcutol CG | Ethoxydiglycol | 5.00 | 5.00 | 5.00 | 5.00 | 5.00 | 5.00 | 5.00 | 5.00 |
| Glycerol, anhydrous | Glycerin | 4.00 | 4.00 | 4.00 | 4.00 | 4.00 | 4.00 | 4.00 | 4.00 |
| Arlasolve DMI | Dimethyl Isosorbide | 2.00 | 2.00 | 2.00 | 2.00 | 2.00 | 2.00 | 2.00 | 2.00 |
| Tween 80 | Polysorbate 80 | 1.60 | 1.60 | 1.60 | 1.60 | 1.60 | 1.60 | 1.60 | 1.60 |
| Aloe CON UP 10 COS. | Aloe Barbadensis | 0.30 | 0.30 | 0.30 | 0.30 | 0.30 | 0.30 | 0.30 | 0.30 |
| Colorants | Colorants | q.s. | q.s. | q.s. | q.s. | q.s. | q.s. | q.s. | q.s. |
| Preservative | Preservative | q.s. | q.s. | q.s. | q.s. | q.s. | q.s. | q.s. | q.s. |
| Water, demineralized | Aqua (Water) | 69.60 | 65.60 | 67.60 | 62.60 | 64.60 | 57.60 | 66.60 | 60.60 |

The entire disclosures of all applications, patents and publications, cited herein and of corresponding German application No. 102007010986.7, filed Mar. 5, 2007 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. An effect pigment comprising a flake-form substrate optionally coated with one or more metal oxides, and having, as final layers,
(A) a layer of iron oxide and/or iron oxohydrate and thereon
(B) a layer of SiO₂ and/or Al₂O₃ or hydrates thereof or
(A) a layer comprising a mixed oxide, hydroxide, oxohydrate or double hydroxide of the general formula M¹ᵥM²_{w}Oₓ(OH)_{y}X_{z}, where
M¹=Fe and M²=Mg^{II}, Ca^{II}, Sr^{II}, Ba^{II}, Ti^{III/IV}, Zr^{IV}, Cr^{III}, Fe^{II/III}, Co^{II/III},
Ni^{II}, Cu^{II}, Ag^{I}, Zn^{II}, Al^{III}, Ga^{III}, Sn^{II/IV}, Sb^{III/IV}, Bi^{III},
X═Cl⁻, NO₃⁻, SO₄²⁻, CO₃²⁻ and
v=1-3; w=0-3; x=0-6, y=0-12 and z=0-1
where v+w>0 and x+y>0, and
v, w, x and y in the case of suboxides may also be non-integer coefficients, and
(B) a layer of SiO₂ and/or Al₂O₃ or hydrates thereof,
and optionally on layer (B) a post coating or post treatment increasing light, water or weather stability.

2. An effect pigment according to claim 1, wherein the coated or uncoated substrate glass flakes, synthetic or natural mica flakes, SiOₓ flakes (x≦2.0), Al₂O₃ flakes, TiO₂ flakes, synthetic or natural iron oxide flakes, optionally passivated metal flakes, flakes of aluminium bronzes, brass bronzes, zinc bronzes, titanium bronzes, graphite flakes, liquid crystal polymers, holographic pigments, BiOCl flakes, or mixtures of said flakes.

3. An effect pigment according to claim 1, wherein the substrate is coated with one, two, three or four metal oxides and the layers (A) and (B).

4. An effect pigment according to claim 1, wherein the substrate is covered with TiO₂ in rutile or anatase modification, zirconium oxide, tin oxide, zinc oxide, or silicon dioxide.

5. An effect pigment according to claim 1, wherein layer (A) consists of Fe₂O₃, Fe₃O₄, FeOOH, FeTiO₃, Fe₂TiO₅ Fe(OH)₃/Al(OH)₃ mixture, Fe(OH)₃/Ca(OH)₂ mixture, Fe(OH)₃/Mg(OH)₂ mixture or Fe(OH)₃/Zn(OH)₂ mixture.

6. An effect pigment according to claim 1, wherein layer (A) has a thickness of 2-350 nm.

7. An effect pigment according to claim 1, wherein layer (B) consists of SiO₂.

8. An effect pigment according to claim 1, wherein layer (B) is a mixture of SiO₂ and Al₂O₃.

9. An effect pigment according to claim 1, wherein layer (B) is doped with oxides or hydroxides of V, Zr, Zn, Ce, Ti, B, Na, K, Mg, Ca and/or Mn.

10. An effect pigment according to claim 1, wherein layer (B) has a thickness of 2-200 nm.

11. An effect pigment according to claim 1, having the following layer sequence:
substrate+(SiO₂)+Fe₂O₃+SiO₂
substrate+(SiO₂)+TiO₂+Fe₂O₃+SiO₂
substrate+(SiO₂)+TiO₂+SiO₂+TiO₂+Fe₂O₃+SiO₂
substrate+(SiO₂)+FeOOH+SiO₂
substrate+(SiO₂)+TiO₂+FeOOH+SiO₂
substrate+(SiO₂)+Fe₂TiO₅+SiO₂
substrate+(SiO₂)+TiO₂+Fe₂TiO₅+SiO₂
substrate+(SiO₂)+FeTiO₃+SiO₂
substrate+(SiO₂)+TiO₂+FeTiO₃+SiO₂
substrate+(SiO₂)+Fe(OH)₃/Mg(OH)₂+SiO₂
substrate+(SiO₂)+TiO₂+Fe(OH)₃/Mg(OH)₂+SiO₂
substrate+(SiO₂)+Fe(OH)₃/Ca(OH)₂—+SiO₂
substrate+(SiO₂)+TiO₂+Fe(OH)₃/Ca(OH)₂+SiO₂
substrate+(SiO₂)+Fe₃O₄+SiO₂
substrate+(SiO₂)+TiO₂+Fe₃O₄+SiO₂
substrate+(SiO₂)+Fe(OH)₃/Al(OH)₃+SiO₂
substrate+(SiO₂)+TiO₂+Fe(OH)₃/Al(OH)₃+SiO₂
substrate+(SiO₂)+TiO₂+FeCa₂(OH)₆Cl+SiO₂
substrate+(SiO₂)+TiO₂+FeCa₂(OH)₆NO₃+SiO₂
substrate+(SiO₂)+TiO₂+FeMg₂(OH)₆CO₃+SiO₂
substrate+(SiO₂)+TiO₂+FeZn₂(OH)₆CO₃+SiO₂
substrate+(SiO₂)+TiO₂+CrO₃+Fe₃O₄+SiO₂
substrate+(SiO₂)+Fe(OH)₃/Zn(OH)₂+SiO₂
substrate+(SiO₂)+Fe(OH)₃/Zn(OH)₂+SiO₂/Al₂O₃
substrate+(SiO₂)+Fe₂O₃+SiO₂+Fe₂O₃+SiO₂/Al₂O₃
substrate+(SiO₂)+Fe₂O₃+SiO₂+Fe₂O₃+SiO₂
substrate+(SiO₂)+Fe₃O₄+SiO₂+Fe₃O₄+SiO₂/Al₂O₃
substrate+(SiO₂)+Fe₃O₄+SiO₂+Fe₃O₄+SiO₂
substrate+(SiO₂)+Fe₂O₃+SiO₂/Al₂O₃
substrate+(SiO₂)+TiO₂+Fe₂O₃+SiO₂/Al₂O₃
substrate+(SiO₂)+TiO₂+SiO₂+TiO₂+Fe₂O₃+SiO₂/Al₂O₃
substrate+(SiO₂)+FeOOH+SiO₂/Al₂O₃
substrate+(SiO₂)+TiO₂+FeOOH+SiO₂/Al₂O₃
substrate+(SiO₂)+Fe₂TiO₅+SiO₂/Al₂O₃
substrate+(SiO₂)+TiO₂+Fe₂TiO₅+SiO₂/Al₂O₃
substrate+(SiO₂)+FeTiO₃+SiO₂/Al₂O₃
substrate+(SiO₂)+TiO₂+FeTiO₃+SiO₂/Al₂O₃
substrate+(SiO₂)+Fe(OH)₃/Mg(OH)₂+SiO₂/Al₂O₃
substrate+(SiO₂)+TiO₂+Fe(OH)₃/Mg(OH)₂+SiO₂/Al₂O₃
substrate+(SiO₂)+Fe(OH)₃/Ca(OH)₂—+SiO₂/Al₂O₃
substrate+(SiO₂)+TiO₂+Fe(OH)₃/Ca(OH)₂—+SiO₂/Al₂O₃
substrate+(SiO₂)+Fe₃O₄+SiO₂/Al₂O₃
substrate+(SiO₂)+TiO₂+Fe₃O₄+SiO₂/Al₂O₃
substrate+(SiO₂)+Fe(OH)₃/Al(OH)₃+SiO₂/Al₂O₃ or
substrate+(SiO₂)+TiO₂+Fe(OH)₃/Al(OH)₃+SiO₂/Al₂O₃,
wherein (SiO₂) represents an optional SiO₂ layer.

12. A process for the preparation of a pigment according to claim 1, comprising coating of a flake-form substrate by wet-chemical methods, by CVD or by PVD methods.

13. In paints, coatings, automobile paints, powder coatings, printing inks, security printing inks, plastics, ceramic materials, glasses, paper, paper coatings, toners for electrophotographic printing processes, seed, in greenhouse sheeting or tarpaulins, absorbers in laser marking of paper and plastics, absorbers in laser welding of plastics, or cosmetic formulations, comprising a pigment, the improvement wherein the pigment is one of claim 1.

14. In day creams, foundations, self-tanning creams, self-tanning sprays or self-tanning lotions comprising a pigment, the improvement wherein the pigment is one of claim 1.

15. A composition comprising (a) a pigment of claim 1, and (b) 1,3-dihydroxyacetone and/or erythrulose.

16. A method of achieving skin correction in cosmetic formulations, comprising combining said formulations with a pigment of claim 1.
